(19) United States
(12) Patent Application Publication
(10) Pub. No.: US 2024/0102922 A1
(43) Pub. Date: Mar. 28, 2024

(54) THREE-DIMENSIONAL DETECTION METHOD AND DEVICE FOR RED TIDE ALGAE BASED ON HOLOGRAPHIC IMAGING

(71) Applicant: ZHEJIANG UNIVERSITY, Hangzhou (CN)

(72) Inventors: Xiaoping WANG, Hangzhou (CN); Kaiqi LANG, Hangzhou (CN); Jiaqing QIANG, Hangzhou (CN); Yuyi QIU, Hangzhou (CN)

(73) Assignee: ZHEJIANG UNIVERSITY, Hangzhou (CN)

(21) Appl. No.: 18/198,229

(22) Filed: May 16, 2023

(30) Foreign Application Priority Data
Sep. 16, 2022 (CN) 202211127801.5

(51) Int. Cl. G01N 21/27; G01N 21/25; G01N 33/18

(52) U.S. Cl. CPC G01N 21/27; G01N 21/255; G01N 33/1826; G01N 2201/06113; G01N 2201/0636

(57) ABSTRACT

A three-dimensional detection method for red tide algae based on holographic imaging is provided, including: constructing a coaxial optical path based on a structure of a Mach-Zehnder interferometer; determining reconstruction distances of the system under different object distances; constructing an original hologram database of the algae; selecting an original hologram, and two reconstructed holograms with different reconstruction distances as a three-dimensional spatial sample of the original hologram; labeling algae in sample, and recording species and bounding boxes of the algae; training a target detection network with the sample of the holograms and a label file; detecting, with the trained target detection neural network, the sample of the algae to obtain species and a quantity of plankton in imaged water. The disclosure further provides a three-dimensional detection device for red tide algae. The method can improve a detection speed and detection accuracy of the red tide algae.

### CROSS REFERENCE TO RELATED APPLICATION

This patent application claims the benefit and priority of Chinese Patent Application No. 202211127801.5 filed with the China National Intellectual Property Administration on Sep. 16, 2022, and entitled “THREE-DIMENSIONAL DETECTION METHOD AND DEVICE FOR RED TIDE ALGAE BASED ON HOLOGRAPHIC IMAGING”, the disclosure of which is incorporated by reference herein in its entirety as part of the present application.

### TECHNICAL FIELD

The present disclosure relates to the technical field of red tide algae monitoring, and relates to a three-dimensional detection method for red tide algae based on holographic imaging and a three-dimensional detection device for red tide algae.

### BACKGROUND

The frequent occurrence of harmful red tide algae has caused severe ecological damage and massive economic losses to marine aquaculture as well. They are monitored through optical microscopy, flow cytometry, fluorescence spectrometry or holographic microscopic imaging at present.

A traditional optical microscope can merely be used for clearly imaging red tide algae on the focal plane due to its small depth of field, resulting in a small sample size and limited information available to each detection.

A flow cytometer is used for detecting cells in fluid. It is likely to produce errors because bubbles, produced by a peristaltic pump, are identified as algae and cell walls of red tide algae are damaged after passing through the peristaltic pump.

The fluorescence spectrometry is merely applicable to algae with required abundance in seawater, and cannot detect algae with low density.

As for the existing holographic microscopic imaging, extraction of a target area, autofocus and classification of algae are performed with complex and time-consuming algorithms, which requires high accuracy of autofocus.

As disclosed in Patent Application Publication No. CN114418995A, a cascading statistical method for algae cells based on a microscope image includes: collecting and labeling algae image sample data, and constructing a deep learning model; training the deep learning model to obtain a deep learning detection model; and identifying labeled image sample data based on the deep learning detection model to obtain an identification result. The method has the problem of undesired identification accuracy since bubbles or impurities probably existing in a sample liquid may be identified as algae cells.

As disclosed in Patent document CN215727660U, a system for rapidly detecting marine red tide based on spectral imaging includes a master controller and a spectral imaging device for imaging a sample culture dish. The spectral imaging device includes an industrial camera, as well as a background plate and a light source in a camera obscura. The sample culture dish is placed at the background plate and irradiated by the light source. The light source includes a halogen light source and an optical filter which may transmit light with relevant characteristic wavelengths of red tide algae at the light input end. The optical filter is mounted on a rotary wheel, and the rotary wheel is driven by a stepping motor. The master controller collects a sample image from the culture dish by controlling the industrial camera and the stepping motor to detect the red tide. In order to identify features of a spectrogram of red tide algae, the method obtains and processes the spectrogram with plenty of time but low accuracy.

### SUMMARY

In order to solve the above problem, the present disclosure provides a three-dimensional detection method for red tide algae based on holographic imaging. The method can improve a detection speed and detection accuracy of the red tide algae.

The three-dimensional detection method for red tide algae based on holographic imaging includes:
step 1: constructing a digital holographic imaging system based on an optical path form of a Mach-Zehnder interferometer;
step 2: taking holograms of different kinds of red tide algae using the digital holographic imaging system, and constructing an original hologram data set of corresponding red tide algae;
step 3: reconstructing the hologram of the red tide alga according to preset reconstruction distances to obtain two corresponding reconstructed holograms with different reconstruction distances, and combining the hologram of the red tide alga with the two corresponding reconstructed holograms to form three-dimensional spatial sampling data;
step 4: processing, based on the reconstruction process in step 3, the original hologram data set of the red tide algae obtained in step 2 to obtain a corresponding three-dimensional spatial sampling data set;
step 5: labeling the red tide algae in the three-dimensional spatial sampling data set to obtain a corresponding label set;
step 6: training a pre-constructed target detection model using the three-dimensional spatial sampling data set in step 4 and the label set obtained in step 5 to obtain a red tide alga detection model for detecting red tide algae; and
step 7: inputting three-dimensional spatial sampling data of the red tide algae to be detected into the red tide alga detection model, and outputting a detection result of the red tide algae to be detected, where the detection result includes species, a quantity and positions of the red tide algae.

According to the present disclosure, the pre-constructed detection model is trained with the three-dimensional spatial sampling data constructed based on the hologram of the red tide alga and the two corresponding reconstructed holograms to obtain the corresponding red tide alga detection model, and the red tide alga detection model analyzes and detects the algae to be detected and outputs the detection result of the red tide algae.

Specifically, the original hologram data set of the red tide algae is obtained by photographing a cuvette having a back wall fixed on a focal plane of an object optical axis of the digital holographic imaging system and having an optical path of 200 μm.

Specifically, in step 3, the reconstruction distances are 0.08 m and 0.16 m respectively.

Specifically, the reconstructed holograms are obtained by computing the hologram of the red tide algae with an angular spectrum reconstruction algorithm.

Specifically, the angular spectrum reconstruction algorithm is specifically expressed as follows:

$$\{ \begin{matrix}U ( x , y , z ) = {- 1}^{} \{ Filter \{ [ H ( x , y ) ] \} \times G ( {f}_{x} . {f}_{y} , z ) \} \\ G ( {f}_{x} . {f}_{y} , z ) = exp [ j \frac{2 π z}{λ} \sqrt{1 - {( λ {f}_{x} )}^{2} - {( λ {f}_{y} )}^{2}} ]\end{matrix} ,$$

where [symbol] represents Fourier transform, [symbol] ⁻¹ represents inverse Fourier transform, Filter{·} represents a frequency filter for obtaining +1 order spectrum and −1 order spectrum of a sample, λ represents a wave field of a light source, z represents a reconstruction distance, and G(fₓ, f_{y}, z) represents an optical transfer function in a frequency domain corresponding to a propagation distance.

Specifically, in step 3, the three-dimensional spatial sampling data are three-dimensional matrix data consisting of the hologram of the red tide algae and the two reconstructed holograms in an ascending order of reconstruction distances.

Specifically, in step 5, the label set includes positions and sizes of bounding boxes and species of the red tide algae, and a position of each bounding box is an XY two-dimensional plane position.

The present disclosure further provides a three-dimensional detection device for red tide algae. The three-dimensional detection device for red tide algae includes a computer memory, a computer processor and a computer program that is stored in the computer memory and executable on the computer processor, where the red tide alga detection model above is stored in the computer memory.

When executing the computer program, the computer processor implements steps including: inputting three-dimensional spatial sampling data of red tide algae to be detected, performing analysis and detection with the red tide alga detection model, and outputting a detection result of the red tide algae to be detected.

Compared with the conventional art, the present disclosure has the following beneficial effects:

according to the present disclosure, it is not necessary to extract and cut an image area of the red tide algae, the original hologram and the two corresponding reconstructed holograms are merely needed to be combined into the three-dimensional spatial sampling data, and the red tide alga detection model obtained through training is used for detection, so as to improve the identification accuracy and the identification speed of the red tide algae.

### BRIEF DESCRIPTION OF THE DRAWINGS

FIG. 1 is a schematic flowchart of a three-dimensional detection method for red tide algae based on holographic imaging according to the present disclosure;

FIG. 2 is a schematic diagram of a coaxial optical path based on a structure of a Mach-Zehnder interferometer according to one embodiment;

FIG. 3 is an enlarged side view of structure A in FIG. 2;

FIG. 4 is a schematic diagram of positions of reconstruction planes of a reconstructed hologram group according to one embodiment; and

FIGS. 5A-5H are holograms of part of red tide algae according to one embodiment.

### DETAILED DESCRIPTION OF THE EMBODIMENTS

In order to facilitate understanding by a person of ordinary skill in the art, a structure of the present disclosure will be further described in detail with reference to embodiments and accompanying drawings.

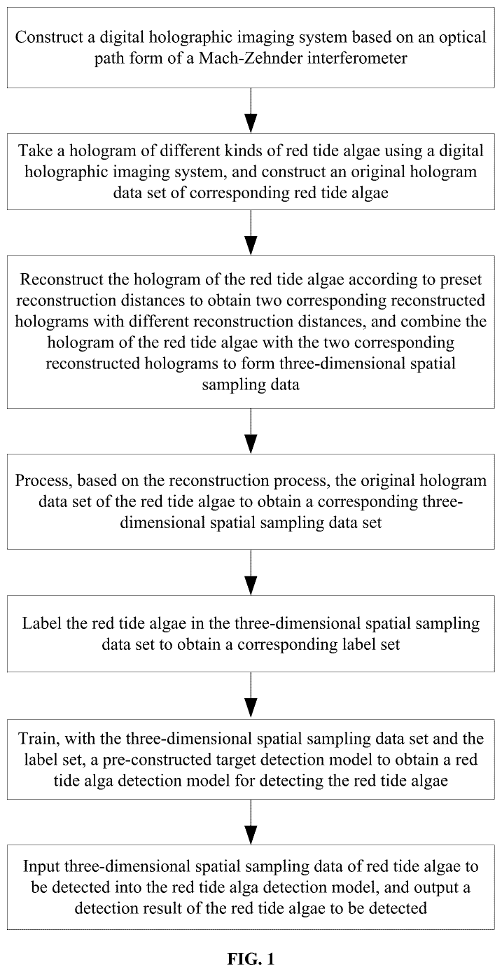

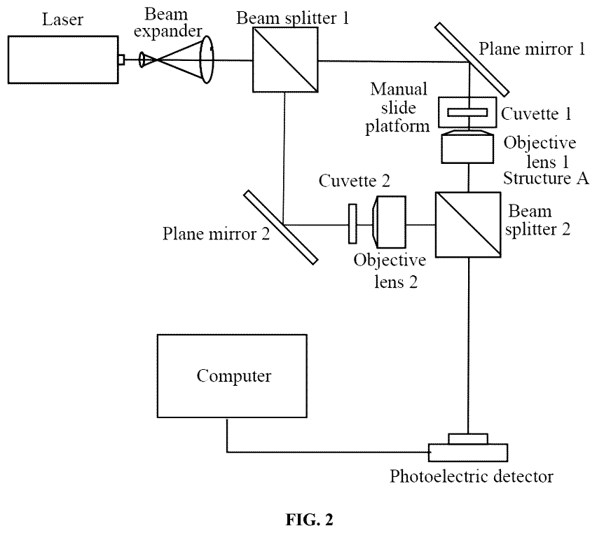

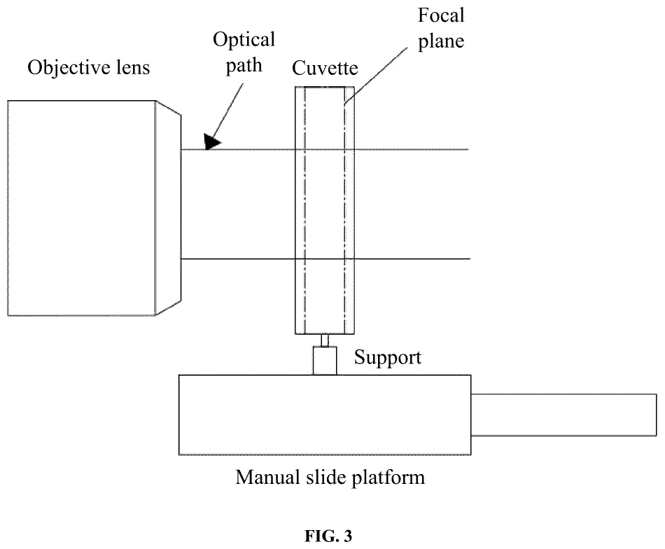

As shown in FIG. 1, a three-dimensional detection method for red tide algae based on holographic imaging includes Steps 1-7:
In Step 1, an optical path is constructed according to a structure of a Mach-Zehnder interferometer shown in FIG. 2. In order to detect micro plankton, a microscopic objective lens is introduced at a back of a sample for amplification to form a digital holographic imaging system.
In Step 2, a resolution plate and a linear slide platform are used to determine a reconstruction distance for a hologram under different object distances. The resolution plate is first fixed on a manual linear slide platform as shown in FIG. 3, a hologram taken by a photoelectric detector is observed on a computer, after a focal plane on an optical axis of an object is determined, the manual linear slide platform is adjusted, a position of the resolution plate is gradually adjusted from the focal plane to 200 μm in front of the focal plane, and holograms are recorded when the resolution plate is at different positions.

Whether the resolution plate in the image is focused is determined artificially, and the reconstruction distance for the hologram in the case of being focused is determined. A cuvette (sample cuvette: a sample put therein) is fixed, instead of the resolution plate, on the manual linear slide platform, as shown in FIG. 3, and an inner wall at a back of the cuvette is fixed on the focal plane. An identical type of cuvette is placed at the same position of a reference arm on the other side (reference cuvette: pure water placed therein).

In the embodiment, an optical path of the sample cuvette is 200 μm and several kinds of red tide algae are selected for experiments, and a hologram of red tide algae is taken to construct an original hologram data set of the red tide algae.
In Step 3, according to the reconstruction distances and the optical path of the cuvette at different object distances obtained in the experiment, as shown in FIG. 4, the original hologram is reconstructed by selecting reconstruction distances of 0.08 m and 0.16 m to obtain two reconstructed holograms, and the hologram is reconstructed according to an angular spectrum reconstruction algorithm:

$$\{ \begin{matrix}U ( x , y , z ) = {- 1}^{} \{ Filter \{ [ H ( x , y ) ] \} \times G ( {f}_{x} . {f}_{y} , z ) \} \\ G ( {f}_{x} . {f}_{y} , z ) = exp [ j \frac{2 π z}{λ} \sqrt{1 - {( λ {f}_{x} )}^{2} - {( λ {f}_{y} )}^{2}} ]\end{matrix} ,$$

where [symbol] represents Fourier transform, [symbol] ⁻¹ represents inverse Fourier transform, Filter {·} represents a frequency filter for obtaining +1 order spectrum and −1 order spectrum of a sample, λrepresents a wave field of a light source, z represents the reconstruction distance, and G(fₓ, f_{y}, z) represents an optical transfer function in a frequency domain corresponding to a propagation distance.

The original hologram and the two reconstructed holograms constitute a three-dimensional matrix in an ascending order based on the reconstruction distances, this three-dimensional matrix is the three-dimensional spatial sampling data of the original hologram, and the matrix is exported and saved in the format of RGB.
In Step 4, based on the reconstruction process in step 3, the original hologram data set of the red tide algae obtained in step 2 are processed to obtain a corresponding three-dimensional spatial sampling data set.
In Step 5, the red tide algae in the three-dimensional spatial sampling data set are labeled with positions and sizes of bounding boxes and species of the red tide algae to obtain a corresponding label set.
In Step 6, the three-dimensional spatial sampling data set and the corresponding label set are combined into a sample set, and the sample set is divided into a training set, a verification set and a test set according to a certain proportion.

The training set is used to train different target detection models, the models are evaluated by the test set, and evaluation parameters are average precision (AP) and recall rate with a formula as follows:

$$recall = \frac{TP}{TP + FN} ; and$$

$$precision = \frac{TP}{TP + FP} ,$$

where TP (true positive) represents the number of samples, which are determined by the model to be positive samples and actually positive samples, FN (false negative) represents the number of samples, which are determined by the model to be false samples but actually positive samples, and FP (false positive) represents the number of samples, which are determined by the model to be positive samples but actually negative samples.

IOU refers to a ratio of an overlapping area of two bounding boxes to a combined area. AP is an evaluation index that comprehensively considers the recall rate and accuracy. IOU and AP are expressed as:

$$IOU = \frac{TB ∩ PB}{TB ⋃ PB} ; and$$

$$AP = {\int }_{0}^{1} p ( t ) dr ( T ) ,$$

where TB represents a real bounding box, PB represents a predicted bounding box, t represents the preset number of systems, and when IOU is greater than t, it is assumed that the model have correctly detected a target, and p(t) represents precision when the recall rate is r(t)

$$F 1 = \frac{2 \times precision \times recall}{precision + recall} ,$$

where F1 represents a final evaluation index.

According to the above evaluation index, the model is evaluated, so as to select a qualified red tide alga detection model.
In Step 7, three-dimensional spatial sampling data of the red tide algae to be detected are input into the red tide alga detection model, and species, a quantity and positions of the red tide algae to be detected are output.

The disclosure further provides a three-dimensional detection device for red tide algae. The three-dimensional detection device for red tide algae includes a computer memory, a computer processor and a computer program that is stored in the computer memory and executable on the computer processor, where the red tide alga detection model above is used in the computer memory.

When executing the computer program, the computer processor implements steps including: inputting three-dimensional spatial sampling data of red tide algae to be detected, performing analysis and detection with the red tide alga detection model, and outputting a detection result of the red tide algae to be detected.

The embodiment adopts common toxic red tide algae which include alexandrium tamarense (AT), chattonella marina (CM), prorocentrum donghaiense (PD), prorocentrum lima (PL), prorocentrum micans (PM), karlodinium veneficum (KV), heterosigma akashiwo (HA) and karenia mikimotoi (KM), and two detection models are selected for test.

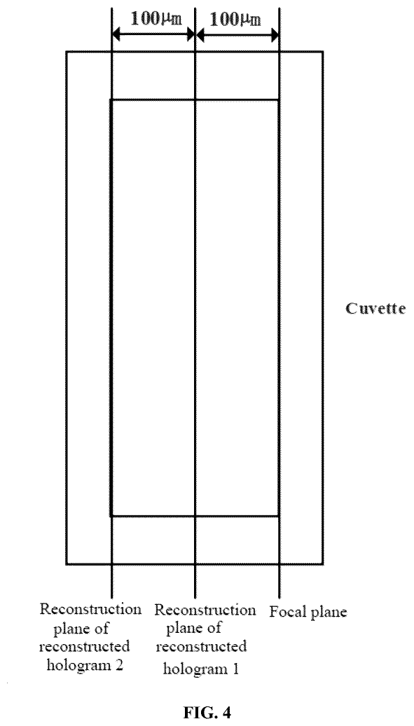

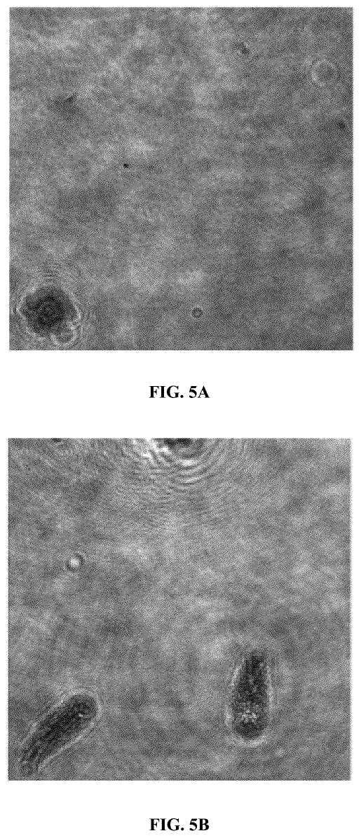

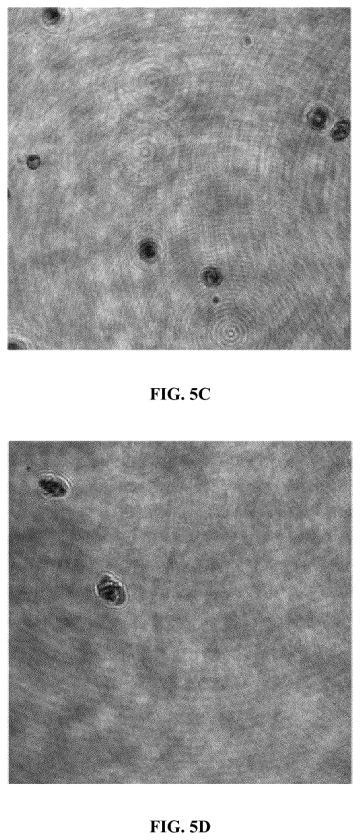

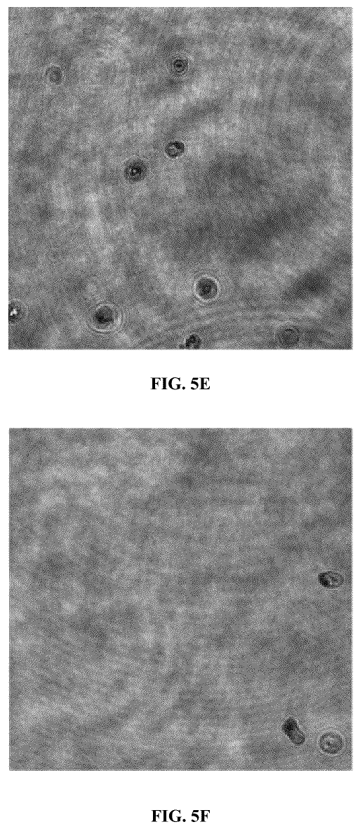

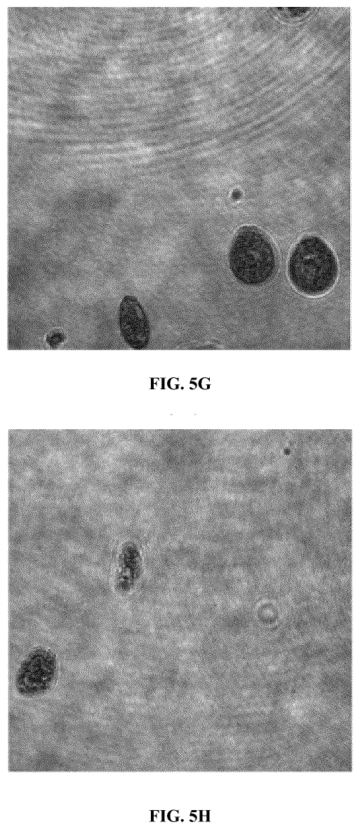

FIGS. 5A-5H show holograms of a part of red tide algae.

A YOLOv4-tiny model and a YOLOv7 model are constructed, and the data set obtained in the above steps is used for training with results obtained shown in Table 1 and Table 2:

TABLE 1
Detection results of YOLOv4-tiny

| Name of red tide algae | AP | Recall | F1 |
|---|---|---|---|
| AT | 99.32 | 98.37 | 0.97 |
| CM | 99.91 | 99.17 | 0.98 |
| HA | 97.19 | 96.50 | 0.93 |
| KM | 98.64 | 98.23 | 0.97 |
| KV | 98.04 | 96.76 | 0.95 |
| PD | 95.84 | 93.67 | 0.94 |
| PL | 99.07 | 99.19 | 0.98 |
| PM | 100.00 | 97.90 | 0.99 |

TABLE 2
Detection results of YOLOv7

| Name of red tide algae | AP | Recall | F1 |
|---|---|---|---|
| AT | 99.99 | 100.00 | 0.99 |
| CM | 100.00 | 100.00 | 0.99 |
| HA | 98.22 | 97.67 | 0.95 |
| KM | 99.59 | 100.00 | 0.97 |
| KV | 99.33 | 99.71 | 0.98 |
| PD | 100.00 | 100.00 | 0.99 |
| PL | 99.27 | 100.00 | 0.97 |
| PM | 100.00 | 100.00 | 1.00 |

For the same graphic data of the red tide algae, time required by the YOLOv4-tiny model

and the YOLOv7 model is as follows:

| Used model | Time required | |
|---|---|---|
| YOLOv4-tiny | 0.086 | s |
| YOLOv7 | 0.12 | s |

In addition, average precision of the above two models reaches 98.5% and 99.55% respectively.

What is claimed is:

1. A three-dimensional detection method for red tide algae based on holographic imaging, comprising:
step 1: constructing a digital holographic imaging system based on an optical path form of a Mach-Zehnder interferometer;
step 2: taking a hologram of different kinds of red tide algae using the digital holographic imaging system, and constructing an original hologram data set of corresponding red tide algae;
step 3: reconstructing the hologram of the red tide algae according to preset reconstruction distances to obtain two corresponding reconstructed holograms with different reconstruction distances, and combining the hologram of the red tide algae with the two corresponding reconstructed holograms to form three-dimensional spatial sampling data;
step 4: processing, based on the reconstruction process in step 3, the original hologram data set of the red tide algae obtained in step 2 to obtain a corresponding three-dimensional spatial sampling data set;
step 5: labeling the red tide algae in the three-dimensional spatial sampling data set to obtain a corresponding label set;
step 6: training a pre-constructed target detection model using the three-dimensional spatial sampling data set in step 4 and the label set obtained in step 5 to obtain a red tide alga detection model for detecting the red tide algae; and
step 7: inputting three-dimensional spatial sampling data of the red tide algae to be detected into the red tide alga detection model, and outputting a detection result of the red tide algae to be detected, wherein the detection result comprises species, a quantity and positions of the red tide algae.

2. The three-dimensional detection method for the red tide algae based on the holographic imaging according to claim 1, wherein in step 2, the original hologram data set of the red tide algae is obtained by photographing a cuvette having a back wall fixed on a focal plane of an object optical axis of the digital holographic imaging system and having an optical path of 200 μm.

3. The three-dimensional detection method for the red tide algae based on the holographic imaging according to claim 1, wherein in step 3, the reconstruction distances are 0.08 m and 0.16 m respectively.

4. The three-dimensional detection method for the red tide algae based on the holographic imaging according to claim 1, wherein in step 3, the reconstructed holograms are obtained by computing the hologram of the red tide algae with an angular spectrum reconstruction algorithm.

5. The three-dimensional detection method for the red tide algae based on the holographic imaging according to claim 4, wherein the angular spectrum reconstruction algorithm is expressed as:
$$\{ \begin{matrix}U ( x , y , z ) = {- 1}^{} \{ Filter \{ [ H ( x , y ) ] \} \times G ( {f}_{x} . {f}_{y} , z ) \} \\ G ( {f}_{x} . {f}_{y} , z ) = exp [ j \frac{2 π z}{λ} \sqrt{1 - {( λ {f}_{x} )}^{2} - {( λ {f}_{y} )}^{2}} ]\end{matrix} ,$$
wherein [symbol] represents Fourier transform, [symbol] ⁻¹ represents inverse Fourier transform, Filter{·} represents a frequency filter for obtaining +1 order spectrum and −1 order spectrum of a sample, λ represents a wave field of a light source, z represents a reconstruction distance, and G(fₓ, f_{y}, z) represents an optical transfer function in a frequency domain corresponding to a propagation distance.

6. The three-dimensional detection method for the red tide algae based on the holographic imaging according to claim 1, wherein in step 3, the three-dimensional spatial sampling data are three-dimensional matrix data consisting of the hologram of the red tide algae and the two reconstructed holograms in an ascending order of reconstruction distances.

7. The three-dimensional detection method for the red tide algae based on the holographic imaging according to claim 1, wherein in step 5, the label set comprises positions and sizes of bounding boxes and species of the red tide algae, and a position of each bounding box is an XY two-dimensional plane position.

8. A three-dimensional detection device for red tide algae, comprising a computer memory, a computer processor and a computer program that is stored in the computer memory and executable on the computer processor, wherein the red tide alga detection model according to claim 1 is stored in the computer memory; and when executing the computer program, the computer processor implements steps comprising: inputting three-dimensional spatial sampling data of the red tide algae to be detected, performing analysis and detection with the red tide alga detection model, and outputting a detection result of the red tide algae to be detected.
